(19) United States
(12) Patent Application Publication
(10) Pub. No.: US 2022/0218826 A1
(43) Pub. Date: Jul. 14, 2022

(54) COMPOSITION FOR TOPICAL USE FOR PHOTODYNAMIC THERAPY

(71) Applicants: Daniela Ines LEON GARRIDO, Temuco (CL); UNIVERSIDAD DE LA FRONTERA, Temuco (CL)

(72) Inventors: Daniela Ines LEON GARRIDO, Temuco (CL); Carmen Gloria ILI GANGAS, Temuco (CL); Priscilla Solange BREBI MIEVILLE, Temuco (CL); Juan Carlos ROA STRAUCH, Santiago (CL)

(21) Appl. No.: 17/611,136

(22) PCT Filed: May 15, 2019
(86) PCT No.: PCT/IB2019/054042

§ 371 Date: Nov. 13, 2021

(51) Int. Cl. A61K 41/00; A61K 47/18; A61K 47/22

(52) U.S. Cl. CPC A61K 41/0061; A61K 47/22; A61K 47/183

(57) ABSTRACT

Composition for photodynamic therapy comprising a photosensitiser in combination with ethylenediaminetetraacetic acid (EDTA) and epigallocatechin galate (EGCG), in pharmacologically acceptable carriers and/or excipients. The photosensitising compounds are selected from protoporphyrin IX (PpIX) precursors, such as methyl aminolevulinate (MAL) or aminolevulinic acid (ALA). These new formulations show an enhancing effect of the effect of the photosensitising compounds, MAL or ALA for example, which ensures a greater efficiency of photodynamic therapy, in the treatment of skin or mucous membranes. In one application, the invention is useful in the resolution of long-term preneoplastic or neoplastic dermatological lesions, decreasing the recurrence rates of these lesions.

### FIELD OF THE INVENTION

The invention is directed to a photosensitising formulation that can be used in topical application for its use in photodynamic therapy (PDT), in the treatment of skin or mucous membranes.

### BACKGROUND OF THE INVENTION

Photodynamic therapy (PDT) is, generally speaking, a type of treatment for hyperproliferative diseases of the skin and internal epithelial, comprising the administration, by topical or systemic route, of a photosensible agent that will ideally be concentrated in the proliferating tissues of the body. The compound itself is inactive but after irradiation with light with a specific wavelength, the molecule is chemically activated and is stimulated for it to undergo chemical reactions that directly damage the cell or result in the production of species that are in turn harmful to the cells. In this way, the chemotherapeutic action is physically limited to an area of interest instead of extending to the whole body of the patient with unpleasant and harmful side effects. The application field of PDT is naturally limited by the accessibility from the tissue to the light source.

Among the hyperproliferative diseases of the skin, cancers can be found where only one of these, melanoma, is seriously life-threatening, and is not a candidate for PDT. Among the non-melanoma skin cancers (NMSC), basal cell carcinoma is found (80% of the cases of NMSC), relatively benign, and squamous cell carcinoma (20% of the cases of NMSC) that have an intermediate danger since they can occasionally metastasize (see World Health Organization. Ultraviolet radiation and the INTERSUN Programme. http://www.who.int/uv/faq/skincancer/en/index1.html (2013)). Hyperplasias, such as actinic keratosis and Bowen's disease, are referred to as precancerous lesions given that these can cause squamous cell carcinoma if not treated.

For this type of lesions or diseases, photodynamic therapy is useful. In practical terms, in order to carry out this dermatological therapy is applied, topically, a formulation comprising a photosensitising (PS) compound, where this compound when irradiated or excited to a light with a specific wavelength generates reactive oxygen species (ROS), which are cytotoxic and induce the death of the cells in which they are found. In dermatology, the common procedure is applying a PS on the skin lesion to be treated, for example, by means of a cream, then letting it incubate and, finally, radiating the area with light for it to generate on-site ROS and the hyperproliferative tissue to be removed.

The invention corresponds to a composition for using in photodynamic therapy (PDT) on the skin, for one of its uses is in the clinical field, specifically, dermatological. This therapy consists of applying the photosensitising cream on the skin lesions and then radiating said lesions with a light of specific wavelength. PDT allows for the treatment of lesions from non-melanoma skin cancer, specifically, basal cell carcinoma and precursor lesions of squamous cell carcinoma of the skin. These lesions have a high incidence worldwide, since these are produced in skin areas exposed to the sun such as, for example, the face. It is estimated that, around the world, there are 2-3 million new cases of this type of cancer each year (see World Health Organization. Ultraviolet radiation and the INTERSUN Programme. http://www.who.int/uv/faq/skincancer/en/index1.html (2013)).

However, some tissues prove to be resistant to this type of therapies (PDT), or the treatment does not adequately reach the whole diseased tissue. For this reason, the development of new compositions that enhance the effect from PSs becomes necessary, this in order to improve the results of the PDT.

In addition, there are other applications for this type of therapy, where the photodynamic therapy is used in the treatment and prevention of inflammatory and infectious diseases of the skin and mucous membranes, and in applications with purely dermocosmetic purposes as well.

In this way, the present invention is related to the pharmaceutical and dermocosmetic industries. Particularly, it is related to a composition for photodynamic therapy that increases its scope, enhancing the effect from known photosensitising compounds, such as the compound methyl aminolevulinate (MAL) or aminolevulinic acid (ALA), for example.

### STATE OF THE ART

In the state of the art are different photosensitisers for photodynamic therapies, where a group of these are protoporphyrin IX (PpIX) precursors, such as methyl aminolevulinate (MAL) or aminolevulinic acid (ALA), which are broadly used for dermatological lesions, such as those that the present invention is directed to.

On 2015, Yang et al. (Yang, X., Palasuberniam, P., Kraus, D., & Chen, B. (2015) International Journal of Molecular Sciences, 16(10), 25865-25880) published that, ferric ion scavengers could enhance the use if this photosensitiser, since said ion is a substrate necessary for the PpIX conversion pathway in hemoglobin, which led to the incorporation of ferric ion chelating agents in photosensitising compositions, in order to improve the therapy. An agent used for this purpose is EDTA.

On the other hand, Mun (Mun S T, Bae D H, Ahn W S. Photodiagnosis Photodyn Ther. 2014. June; 11(2):141-7.) suggests that PDT combined with EGCG could be useful for an effective cancer treatment. Due to the fact that it was observed both a decrease in the TC-1 cell line growth (derived from hybridoma) and from the tumors from this line generated in C57BL/6 mice, after having treated them with PDT+EGCG using Radachlorin as photosensitiser. In this work, EGCG is not applied directly with the photosensitiser in the in vivo assays as is carried out in the invention, but rather was administered by injection in the tumors by 20 days after PDT, with which they observed the best results regarding the decrease in the size of the tumors in mice. Therefore, the results obtained are based on a treatment where EGCG is applied after PDT, not simultaneously with the photosensitiser.

Although these therapies already have several years of development, complete response rates to the treatment have been reported which vary from a 37% to 89%, according to the type of lesion treated and follow-up time to therapy. Consequently, there is a significant percentage of recurrence or resistance to the treatment by PDT in this type of lesions, even with this knowledge available.

### Solution to the Technical Problem

To remedy the problem raised, the inventors have developed a new pharmaceutical and dermocosmetic composition for topical use prepared based on an protoporphyrin IX (PpIX) photosensitiser, for example, methyl aminolevulinate (MAL) or aminolevulinic acid (ALA), combined with ethylenediaminetetraacetic acid (EDTA) and epigallocatechin galate (EGCG). These new formulations show an enhancing effect of the effect of the photosensitising compounds, MAL or ALA, for example, which ensures a greater efficiency of photodynamic therapy, for example, in the resolution of long-term dermatological lesions or of preneoplastic or neoplastic mucous membranes, for example, preneoplastic lesions of the cervix (intraepithelial lesions of the cervix) decreasing the recurrence rate of these lesions. Or in any other application of photodynamic therapy, such as the treatment or prevention of inflammatory diseases, on the skin or mucous membranes, for example, acne, rosacea, cellular rejuvenation, or infectious diseases on the skin or mucous membranes.

### DESCRIPTION OF THE DRAWINGS

FIG. 1. Effect of EDTA and EGCG on MAL-PDT on the viability of PDT-resistant HSC-1 cells assessed by MTT. A) Effect of EGCG associated to PDT. B) Effect of EDTA associated to PDT. C) and D) Controls without light of EGCG and EDTA, respectively. The results represent the ±DS average. The experiments were carried out in technical and biological triplicate. *P<0.05.

FIG. 2. Effect of the formulation from the invention, that combines EDTA and EGCG on MAL-PDT on the viability of PDT-resistant HSC-1 cells assessed by MTT. The results represent the ±DS average. The experiments were carried out in technical and biological triplicate. *P<0.05.

FIG. 3. Detection of protoporphyrin IX (PpIX) in PDT-resistant HSC-1 cells by flow cytometry. A) Effect of EGCG in the PpIX synthesis. B) Effect of EDTA in the PpIX synthesis. The experiments were carried out in technical and biological triplicate. **P<0.005.

FIG. 4. Detection of reactive oxygen species (ROS) in PDT-resistant HSC-1 cells by flow cytometry. A) Effect of EGCG in the generation of ROS. The experiments were carried out in technical and biological triplicate. *P<0.005.

### DESCRIPTION OF THE INVENTION

Photodynamic therapy, as already mentioned, consists of the application of a cream on the skin lesion and then radiated with a specific light. The cream contains a photosensitiser that enters the cell and is accumulated in it. When the lesion is exposed to the specific light, the photosensitiser reacts with the O₂ present in the cell and forms reactive oxygen species that cause cell damage, and consequently cell death. It is an outpatient treatment, which can be applied over large areas of the skin but, above all, its greatest advantage is that optimum cosmetic results are obtained, which is important, considering that many times lesions are developed in areas exposed to the sun. This type of therapies has multiple applications, for example, in lesions of the skin or non-melanoma neoplastic or preneoplastic mucous membranes, in addition this therapy is also employed in the treatment and prevention of inflammatory diseases, on the skin or mucous membranes, for example, acne, rosacea, cellular rejuvenation, or infectious diseases on the skin or mucous membranes.

However, this therapy is not always effective, for example, in non-melanoma neoplastic or preneoplastic lesions, there is a significant rate of recurrences or resistance to the treatment by PDT in this type of lesions.

The invention is directed to new photosensitising pharmaceutical and dermocosmetic formulations of topical application for its use in photodynamic therapy (PDT), based on PpIX precursors, photosensitisers selected from methyl aminolevulinate (MAL) and/or aminolevulinic acid (ALA), combined with ethylenediaminetetraacetic acid (EDTA) and epigallocatechin galate (EGCG). This new combination will ensure a greater efficiency of photodynamic therapy, especially in the treatment of non-melanoma skin cancer, in the resolution of preneoplastic or long-term neoplastic dermatological lesions, decreasing the recurrence rate of these lesions.

The inventors have included in the composition of the invention, EDTA, given that this compound has a chelating effect that contributes to the increase of the cytotoxic effect allowing the PpIX cellular accumulation (compound synthesized by the cells from MAL and ALA), thus increasing the production of reactive oxygen species (ROS) that cause cell damage.

On the other hand, the inventors have included EGCG, given that its presence in the composition boosts and strengthens the efficiency of PDT, has a chelating, prooxidant, and antiproliferative effect. In this way, the composition of the invention allows for PDT to improve its efficiency ensuring the destruction of preneoplastic and neoplastic cells of non-melanoma skin cancer.

Surprisingly, these new combinations have a highly superior effect to that produced by the photosensitiser (ALA or MAL) without the enhancers, which allows for the improvement of the efficiency of the therapy and decrease of recurrence of already treated lesions.

In a preferred embodiment, the composition contains the photosensitiser (MAL or ALA) at a concentration between 100 mg/g-200 mg/g, EDTA at a concentration between 0.5 mg/g-10 mg/g and EGCG at a concentration between 0.1-500 mg/g, in pharmacologically acceptable carriers and/or excipients.

Where photosensitisers, MAL or ALA can be found freely or from a pharmacologically acceptable salt, such as for example, methyl aminolevunilate hydrochloride, aminolevunilic acid methyl ester or methyl 5-amino-4-oxopentanoate hydrochloride, among others.

The excipients are selected from water, sodium chloride, lanolin, beeswax, glycerol, petroleum jelly, propylene glycol, sodium lauryl sulfate, dimethyl sulfoxide, imidazolidinyl urea, olivem 1000, propyl parahydroxybenzoate, Polawax, methylparaben, almond oil, castor oil, cetyl alcohol, butylhydroxytoluene, and any other excipient available in the art.

In addition, the composition of the invention may contain other active compounds or formulation aids.

The composition of the invention may be employed in any application of photodynamic therapy that there is in the art, for example, in pharmaceutical or dermocosmetic applications.

In an embodiment, the composition of the invention may be employed to prepare a drug product useful for photodynamic therapy, useful in the treatment of preneoplastic and neoplastic cells of non-melanoma skin cancer. In a preferred embodiment, the composition of the invention is employed in the treatment of photodynamic therapy-resistant cells or lesions.

In addition, the composition of the invention may be employed in the treatment or prevention of inflammatory and/or infectious diseases on the skin or mucous membranes, for example, acne or rosacea, among others

In another embodiment, the composition of the invention may be employed in photodynamic therapy for dermocosmetic purposes, for example, for cellular rejuvenation.

The composition of the invention may be constituted in different pharmaceutical or dermocosmetic presentations, such as cream, ointment, spray, lotion, foam, or any other existing in the art.

The scope of the invention will become clearer in light of the examples given, which should be considered as illustrative, and under no circumstance limiting of the present invention.

### APPLICATION EXAMPLES

### Example 1

### In Vitro Effect of EGCG and EDTA Separately as Adjuvants of MAL-PDT

The in vitro effect of EGCG and EDTA separately as adjuvants of MAL-PDT was assessed HSC-1 cells derived from squamous skin carcinoma were used. Previously, these cells received PDT cycles, in order to select those that are resistant to PDT. Therefore, the model used corresponds to MAL-PDT-resistant HSC-1 cells.

The in vitro photodynamic conventional treatment consisted of incubating these cells with MAL 150 mg/g (photosensitiser) for 4 hours in the dark and after radiating them with 630 nm red light, with fluence of 4 J/cm². Cell viability was assessed 24 hours later by the MTT assay.

Under these MAL-PDT conditions, which are the control conditions, as a result it was obtained that approximately a 50% of HSC-1 resistant cells survived PDT. Subsequently, this protocol was carried out in the same way in these cells, only with the following modification: when adding MAL, different concentrations of EDTA were added (0.5 mg/g, 1 mg/g, 1.5 mg/g, and 2 mg/g) or EGCG (0.2 mg/g, 0.4 mg/g, 0.6 mg/g, 0.8 mg/g). The MAL concentration of 150 mg/g was kept in all tests and experiments with irradiation (light) fluency same to the control at 4 J/cm², or without radiation, were carried out.

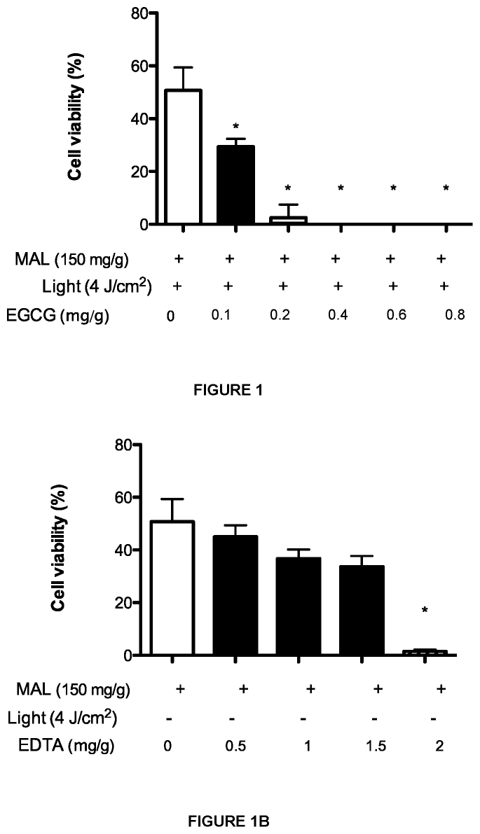

The effect of EGCG on MAL-PDT is observed in FIG. 1A (light) and 1C (controls without light). In all tested EGCG concentrations, under light conditions, the viability of resistant cells decreased significantly, even from 0.4 mg/g, the survival rate was 0. In turn, the effect of EDTA only at a 2 mg/g concentration in MAL-PDT was significant, the results are plotted in FIG. 1B (light) and 1D (controls without light).

### Example 2

### In Vitro Effect of EGCG and EDTA Combined as Adjuvants of MAL-PDT

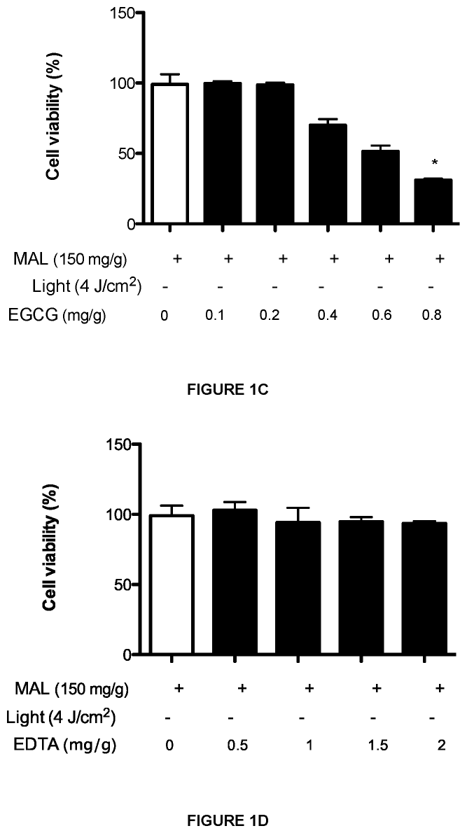

The synergetic effect of the combination of the invention EGCG+EDTA in MAL-PDT was assessed similarly to that discussed in example 1 but with lower EGCG concentrations. In FIG. 2, the effect of 0.5 mg/g, 1 mg/g, and 1.5 mg/g EDTA, in presence of 0.1 mg/g EGCG, as MAL-PDT enhancers in HSC-1 resistant cells, was shown.

The results show that all compositions of the invention had a very significant decrease regarding the control. In the lowest concentrations assessed (10 mg/g EGCG, 0.5 mg/g EDTA, 150 mg/g MAL), cell viability is of only 10%, and under the other 2 conditions there is a 0% viability of MAL-PDT-resistant cells.

### Example 3

Detection of Protoporphyrin IX (PpIX) in PDT-Resistant HSC-1 Cells when Incubated with MAL and EGCG or EDTA

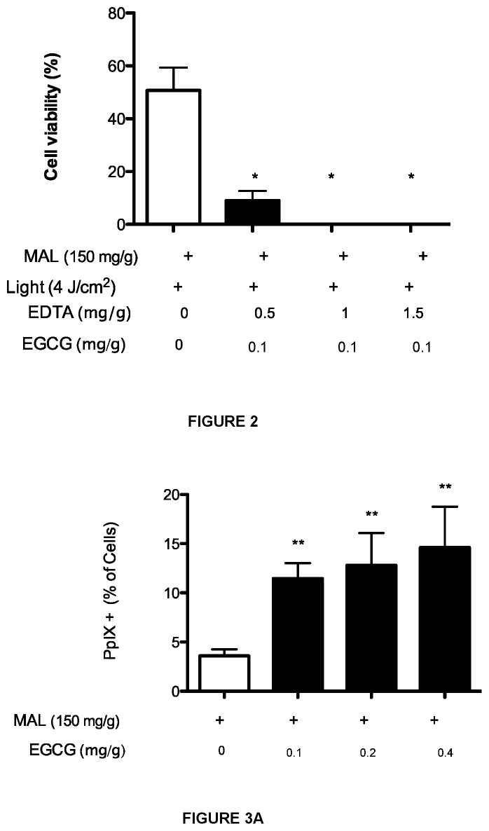

In order to understand the mechanisms of action of the compositions of the invention and, due to the fact that EGCG and EDTA have chelating capability, it was assessed if the presence of these compounds would increase the content of PpIX in cells. For this, PDT-resistant cells, obtained as stated in example 1, were incubated with 150 mg/g MAL, plus EGCG (0.1 mg/g, 0.2 mg/g, 0.4 mg/g) or EDTA (1 mg/g, 2 mg/g, 3 mg/g) for 4 hours in the dark. Subsequently, the PpIX content in resistant cells was detected by flow cytometry, since PpIX is a fluorescent compound. The results show that both compounds significantly increase the production of PpIX. As it can be clearly observed in FIG. 3, when incubating PDT-resistant cells with MAL, only a 5% of the population contained PpIX, while in presence of EDTA (FIG. 3B) or EGCG (FIG. 3A) this percentage increases up to a 13 or 18%, respectively.

### Example 4

Detection of Reactive Oxygen Species (ROS) in PDT-Resistant HSC-1 Cells when Incubated with MAL and EGCG

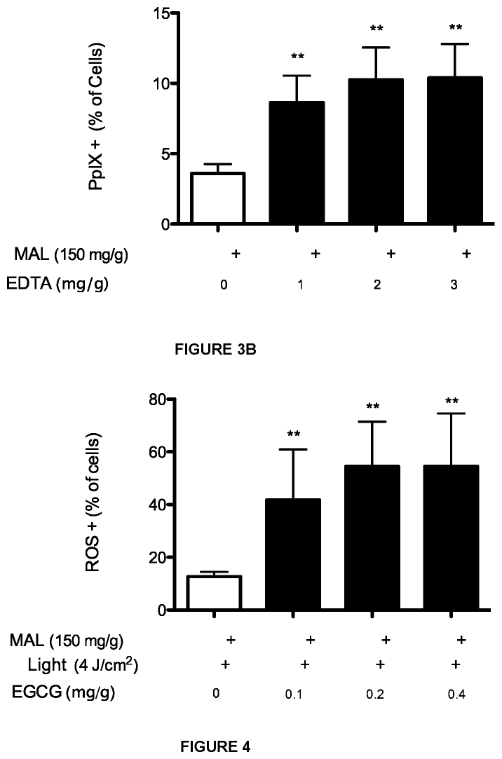

Given that in PDT, it is finally the reactive oxygen substances that cause cell damage and death, the production of these compounds was directly assessed. The same model as in example 3 was used, at the same EGCG concentrations stated in FIG. 3A, in order to assess the production of ROS generated in MAL-PDT. Following the previous methodology, after incubating the cells for 4 hours, these received red light irradiation. For ROS detection, these were incubated with a probe (Muse™ Oxidative Stress Kit|MCH100111—Merck Millipore) for 30 minutes and was assessed in a cell analyzer. The obtained results show that the presence of EGCG in MAL-PDT stimulates the productions of RROS in cells, given that this compound gas a prooxidant effect (FIG. 4). In all assessed EGCG concentrations, the ROS concentration doubled or even tripled the concentration obtained in the control.

Therefore, based on the results described in the examples above, it is shown that the compositions of the invention containing EGCG and EDTA enhance the cytotoxic effect of MAL-PDT in HSC-1 cells resistant to the treatment. On the other hand, the synergetic effect of both compounds may be observed even when using very low concentrations of EGCG, due to the fact that the effect of EGCG is greater than that of EDTA.

Statistical Analysis: the data was presented as the ±DS average and the significance was tested with the Mann Whitney test using GraphPad Prism (GraphPad Software, La Jolla, Calif., USA). All assays were carried out using technical and biological triplicate. The statistical significance was established with a P value <0.05.

1. A composition for photodynamic therapy, comprising a photosensitiser at a concentration between 100 mg/g-200 mg/g combined with ethylenediaminetetraacetic acid (EDTA) at a concentration between 0.5 Mg/g-10 mg/g and epigallocatechin galate (EGCG) at a concentration between 0.1 mg/g-500 mg/g, in pharmacologically acceptable carriers and/or excipients.

2. The composition according to claim 1, wherein the photosensitiser is selected from protoporphyrin IX (PpIX) precursors.

3. The composition according to claim 2, wherein the protoporphyrin precursor photosensitiser is selected from methyl aminolevulinate (MAL) and/or aminolevulinic acid (ALA) and its pharmacologically acceptable salts.

4. The composition according to claim 1, wherein the pharmacologically acceptable carriers and/or excipients are for a topical application and are selected from the group consisting of water, sodium chloride, lanolin, beeswax, glycerol, petroleum jelly, propylene glycol, sodium lauryl sulfate, dimethyl sulfoxide, imidazolidinyl urea, olivem 1000, propyl parahydroxybenzoate, Polawax, methylparaben, almond oil, castor oil, cetyl alcohol, and butylhydroxytoluene.

5. The composition according to claim 4, wherein the composition is in the form of a cream, ointment, spray, lotion, or foam.

6. The composition according to claim 1, wherein the composition is used for preparing a drug product for photodynamic therapy.

7. A method for treating preneoplastic and neoplastic cells of non-melanoma skin cancer comprising using the drug product of claim 6.

8. A method for treating cells or lesions resistant to photodynamic therapy comprising using the drug product of claim 6.

9. A method for treating inflammatory diseases on the skin or mucous membranes comprising using the drug product of claim 6.

10. The method according to claim 9, wherein the inflammatory diseases is acne, or rosacea.

11. A method for treating infectious diseases on the skin or mucous membranes comprising using the drug product of claim 6.

12. A composition for photodynamic therapy for dermocosmetic purposes comprising the composition according to claim 1.

13. The composition according to claim 12, wherein the dermocosmetic composition is useful for cellular rejuvenation.
